(19) United States
(12) Patent Application Publication
(10) Pub. No.: US 2026/0276546 A1
(43) Pub. Date: Sep. 17, 2026

(54) SINGLE EXTRACELLULAR VESICLE MONITORING IN CRUDE CULTURES

(71) Applicant: UNIVERSITY OF NOTRE DAME DU LAC, South Bend, IN (US)

(72) Inventors: Abigail A. WEAVER, South Bend, IN (US); Paul W. BOHN, South Bend, IN (US); Jarek James METRO, South Bend, IN (US)

(21) Appl. No.: 19/567,863

(22) Filed: Mar. 16, 2026

Related U.S. Application Data
(60) Provisional application No. 63/773,062, filed on Mar. 17, 2025.

(51) Int. Cl. G01N 21/64; G01N 33/58

(52) U.S. Cl. CPC G01N 21/6452; G01N 21/6428; G01N 33/582; G01N 2021/6439; G01N 2021/6484; G01N 2333/21; G01N 2333/31

(57) ABSTRACT

Described herein is a zero-mode waveguide (ZMW) array for monitoring populations of single extracellular vesicles in a crude culture in real-time. The ZMW array comprises a plurality of ZMW nanopores arranged in parallel, each ZMW nanopore being configured to hold a single extracellular vesicle. Also described herein are methods and kits for monitoring populations of single extracellular vesicles in crude cultures using ZMW arrays.

### CROSS-REFERENCE TO RELATED APPLICATIONS

This application claims priority to U.S. Provisional Patent Application No. 63/773,062 filed on Mar. 17, 2025, which is incorporated by reference herein in its entirety.

### FEDERALLY SPONSORED RESEARCH

This invention was made with government support under grant R01 AI113219 awarded by the National Institutes of Health (NIH) and grant CHE2303574 awarded by the National Science Foundation (NSF). The government has certain rights in the invention

### BACKGROUND

Extracellular vesicles (EVs) are produced by organisms across all domains of life with varied functions, including intercellular communication, immune response, and properties that reflect the physiological state and environment of the cells from which they originated. Bacterial EVs (bEVs) are well documented to play roles in bacterial cell-to-cell communication and host-pathogen interactions, in addition to their potential as vaccines and vaccine adjuvants. These vesicles also contribute to antimicrobial resistance for certain drugs by acting as decoys and sequestering antibiotics and antimicrobial peptides, transferring resistance genes across cells, and degrading antibiotics through proteins associated with the bEV. These bEVs are derived from cells and range in size from 20-800 nm, formed principally through blebbing of membranes or through explosive cell lysis, with both mechanisms contributing to their diversity of size, lipid composition, and cargo. The opportunistic pathogen Pseudomonas aeruginosa produces bEVs that contain multiple virulence factors and lipopolysaccharides (LPS) that induce an immune response in humans causing inflammation and promoting pathogen clearance. However, dysregulation of this response can cause an exaggerated immune response, increased inflammation, organ damage, sepsis, and even death. Because bEVs play significant roles in various physiological pathways of all organisms and exhibit a broad range of structure and function, analytical methods that can rapidly characterize heterogeneity within and among bEV populations are highly desirable.

Many methods used for EV analysis, such as western blotting and enzyme-linked immunosorbent assays, produce population-averaged measurements that do not provide information about the heterogeneity of the population. Single-particle characterization of EVs has been demonstrated using methods such as Raman tweezers microspectroscopy, atomic force microscopy, and cryo-electron microscopy, but these techniques examine single particles one-at-a-time. They are, thus, low throughput, and they require substantial sample purification and/or preparation. EV isolation and concentration is most often carried out using filtration and differential ultracentrifugation; however, these methods are time-consuming and prone to contamination by non-vesicle components, such as protein aggregates and lipoproteins. Some high throughput, single-vesicle methods have been developed, primarily utilizing immunosorbent assays for fluorescence staining of specific proteins on the surface of the EV using droplet-based extracellular vesicle analysis (DEVA) and using microchamber arrays of antibody barcodes. While these methods can achieve high-throughput, single-vesicle analysis and avoid EV isolation and concentration steps, they are not suitable for in situ monitoring of crude samples as they require significant preparation for the immunosorbent assays, limiting the ability to carry out EV detection in complex samples in real-time.

What is needed are biocompatible devices and methods for in situ detection and monitoring of single EVs from crude cultures in real-time.

### SUMMARY

One embodiment described herein is a zero-mode waveguide (ZMW) array for monitoring populations of single extracellular vesicles in a crude culture, the ZMW array comprising a plurality of ZMW nanopores arranged in parallel, each ZMW nanopore being configured to hold a single extracellular vesicle. In one aspect, each ZMW nanopore has a conical frustum shape with a top diameter that is greater than a bottom diameter. In another aspect, the top diameter is about 250 nm to about 350 nm and the bottom diameter is about 150 nm to about 250 nm. In another aspect, the top diameter is about 300 nm and the bottom diameter is about 200 nm. In another aspect, each ZMW nanopore is configured to restrict entry of particles greater than about 300 nm in size. In another aspect, each ZMW nanopore is configured to hold attoliter (10⁻¹⁸ L) to zeptoliter (10⁻²¹ L) volumes. In another aspect, the ZMW array comprises one or more layers comprising titanium, platinum, palladium, gold, glass, silica, or combinations thereof. In another aspect, the ZMW array comprises greater than 400 ZMW nanopores. In another aspect, the ZMW array comprises an average interpore spacing distance of about 1.5 μm to about 2.5 μm. In another aspect, the ZMW array has dimensions of about 40 μm×40 μm.

Another embodiment described herein is a method for monitoring single extracellular vesicles in a crude culture, the method comprising labeling single extracellular vesicles in the crude culture with a lipophilic fluorophore; contacting the crude culture with a zero-mode waveguide (ZMW) array comprising a plurality of ZMW nanopores arranged in parallel, each ZMW nanopore being configured to hold a single extracellular vesicle, wherein single extracellular vesicles diffuse into the ZMW nanopores; and detecting fluorescence from single extracellular vesicles positioned within the ZMW nanopores. In one aspect, the crude culture comprises a Gram-positive bacterium, a Gram-negative bacterium, or a combination thereof. In another aspect, the crude culture comprises Pseudomonas aeruginosa or Staphylococcus aureus. In another aspect, the extracellular vesicles have an average size of about 200 nm to about 300 nm. In another aspect, the lipophilic fluorophore comprises 3,3′-dioctadecyloxacarbocyanine (DiO), 1,1′-dioctadecyl-3,3,3′, 3′-tetramethylindocarbocyanine perchlorate (DiI), 3,3′-dioctadecyloxacarbocyanine perchlorate, 1,1′-dioctadecyl-3,3,3′, 3′-tetramethylindodicarbocyanine perchlorate (DiD), 1,1′-dioctadecyl-3,3,3′, 3′-tetramethylindotricarbocyanine iodide (DIR), PKH26, PKH67, Nile Red, BODIPY-based lipophilic dyes, or combinations thereof. In another aspect, single extracellular vesicles positioned within the ZMW nanopores have an average residence time of about 2 s to about 5 s. In another aspect, detecting fluorescence comprises irradiating the ZMW array and performing fluorescence microscopy. In another aspect, fluorescence microscopy comprises wide-field fluorescence microscopy, epi-illumination microscopy, or a combination thereof. In another aspect, single extracellular vesicles only fluoresce upon irradiation when positioned within the ZMW nanopores. In another aspect, contacting comprises adding the crude culture to the ZMW array and applying an electrokinetic force across the ZMW array. In another aspect, the method further comprises treating the crude culture with one or more agents and monitoring changes in fluorescence detection. In another aspect, the one or more agents comprise an antibiotic. In another aspect, bacteria in the crude culture are restricted from entering the ZMW nanopores based on size. In another aspect, a plurality of single extracellular vesicles are detected and monitored simultaneously in real-time. In another aspect, the method does not include isolating extracellular vesicles from the crude culture prior to contacting the crude culture with the ZMW array.

Another embodiment described herein is a kit for monitoring single extracellular vesicles in a crude culture, the kit comprising a zero-mode waveguide (ZMW) array comprising a plurality of ZMW nanopores arranged in parallel, each ZMW nanopore being configured to hold a single extracellular vesicle; a detection reagent; optionally, buffers and receptacles; and optionally, one or more of packaging, a label, or instructions for use. In one aspect, the detection reagent comprises one or more lipophilic fluorophores for labeling extracellular vesicles. In another aspect, the kit further comprises a detection means. In another aspect, the detection means comprises a fluorescence detection and imaging device.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-D show zero mode waveguide characterization and detection scheme. FIG. 1A shows a plan view scanning electron microscope (SEM) image of a portion of the zero-mode waveguide (ZMW) array surface. FIG. 1B shows a cross-sectional SEM image of a single nanopore. FIG. 1C shows a widefield image with DiO fluorescence from EVs emanating from ZMW nanopores. FIG. 1D (Top) shows signal intensity acquired from a single pore over 60 s. FIG. 1D (Bottom) shows a scheme depicting the spatial location of EVs in the OFF state before entering the nanopore (i), in the ON state upon entering the optical volume (ii) and re-entering the OFF state upon leaving the nanopore (iii).

FIG. 2A-E show the effect of sample preparation on EV behavior in ZMWs. FIG. 2A shows a schematic illustration of stages of EV sample preparation prior to application to ZMWs. FIG. 2B shows peak picking process for a single representative nanopore. The raw signal is smoothed using a moving average, and peaks are found using a threshold. FIG. 2C shows relative frequency of EV detection events per pore. FIG. 2D shows relative frequency of peak widths, t_{width}, in bacterial cultures, supernatant, and filtrate. FIG. 2E shows histograms of (i) average events per pore, and (ii) peak widths. Significance was determined using (i) two-tailed t-test and (ii) Mann-Whitney U test. Error bars represent the standard error of the mean. *p≤0.05, ***p≤0.001.

FIG. 3A-B show EV production from mutant and wild-type (WT) P. aeruginosa. FIG. 3A shows Relative size distributions of EVs from P. aeruginosa WT and the Δ pqsA mutant, which is deficient in blebbing-induced EV formation. FIG. 3B shows average (i) events per pore and (ii) t_{width}. Significance was determined using a (i) two-tailed T-test and (ii) Mann-Whitney U test. Error bars represent standard error of the mean. ***p≤0.001.

FIG. 4A-B show the effect of ciprofloxacin on real-time, in situ detection of EV formation. FIG. 4A shows distributions of numbers of events across the entire nanopore array at various time points after injection of solution without ciprofloxacin (negative control). The bar represents the average for each distribution, and the error bars show the standard error of the mean. FIG. 4B shows distributions of numbers of events across the entire nanopore array distribution at various time points after injection of ciprofloxacin. Width of violin figures represents the relative number of occurrences. **p≤0.01, ****p≤0.0001.

FIG. 5 shows an illustration of an exemplary ZMW array and method as described herein for detecting and monitoring single EVs in crude culture from Pseudomonas aeruginosa.

FIG. 6 shows an exemplary ZMW fabrication. (1) An adhesion layer of 10 nm Ti followed by 100 nm Au and an additional 5 nm Ti is deposited onto a cleanroom-cleaned glass slide via electron-beam evaporation. (2) 100 nm SiO₂ is deposited on top of the metallic layers via plasma-enhanced chemical vapor deposition. (3) Nanopores are milled into the stack using FIB milling, resulting in an array of 441 ZMWs.

FIG. 7 shows a schematic diagram of the fluorescence microscopy apparatus. Widefield microscopy using 458 nm radiation was implemented in an epi-illumination configuration, and DiO fluorescence was collected through a 100× objective, passed through a dichroic mirror, and relayed to an EMCCD detector.

FIG. 8A-B show dynamic light scattering (DLS) of P. aeruginosa samples and the effect of filtration on bEV size distributions. FIG. 8A shows DLS measurements of relative abundance in bacterial culture supernatant after centrifugation and filtering. The average bEV diameters for the supernatant and filtrate were 276±9 nm and 247±9 nm, respectively. FIG. 8B shows deconvolution of the supernatant size distribution into three gaussian components. The fit of the cumulative distribution yields three distinct peak populations centered near 150 nm (1), 350 nm (2), and 600 nm (3).

FIG. 9A-B show characteristics of bEVs obtained from Gram-positive and Gram-negative bacteria. FIG. 9A shows the number of events per pore for S. aureus and P. aeruginosa. FIG. 9B shows the average t_{width} for each filtered culture. Samples were passed through 0.45 μm filters one time. The error bars represent the standard error of the measurement.

### DETAILED DESCRIPTION

The present disclosure relates generally to zero-mode waveguide (ZMW) arrays and methods for detecting and monitoring populations of single extracellular vesicles (EV) in crude cultures. EVs have emerged as key factors for intercellular communication, disease biomarkers, and vaccines, but EV populations generally exhibit broad heterogeneity, making single-vesicle measurements critical to understand the roles played by EVs and the pathways they utilize. To circumvent the exhaustive isolation and concentration protocols and the long incubation periods required by common single-vesicle characterization methods, the ZMW arrays and methods described herein were developed for in situ studying of single EVs from crude culture in real-time with minimal sample preparation.

In various aspects, the disclosed ZMW array comprises a plurality of ZMW nanopores arranged in parallel. The dimensions of each ZMW nanopore are configured to hold and be occupied by only a single EV, making it possible to monitor large arrays of single EVs one-at-a-time in parallel. Furthermore, the attoliter-to zeptoliter-volume of the ZMW nanopores restricts much larger cells and debris in the crude culture (e.g., bacterial cells) from entering the observation volume, eliminating the need to isolate EVs from their parent cells.

In various aspects, detection reagents (e.g., lipophilic fluorophores) are used to selectively tag EV membranes, thereby restricting optical observations to single EVs captured one-at-a-time in individual ZMW nanopores. The minimal sample preparation and the ability to monitor the crude culture sample in situ enables real-time analysis of changes in the crude culture environment, as the detection and monitoring of EVs is governed by the diffusion of single EV particles into the ZMW nanopore optical volume. The arrays and methods disclosed herein provide an improved approach for characterizing EV heterogeneity in crude cultures and enable real-time observation of shifts in the vesicle population in response to culture perturbations.

### Definitions

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “zero mode waveguide nanopore” or “ZMW nanopore” means a photonic nanostructure comprising a sub-wavelength aperture having lateral dimensions below the cutoff for far-field propagation of optical modes, thereby confining illumination to sub-wavelength dimensions and attoliter-to zeptoliter-scale observation volumes. ZMW nanopores are formed at the base of the sub-wavelength aperture, enabling entry and optical interrogation of single entities, e.g., molecules, particles, vesicles, in the confined volume and optical field.

As used herein, the term “top of the nanopore” or “top diameter” refers to the ZMW nanopore opening that is in contact with the sample-containing fluid solution. In addition, as used herein, the term “bottom of the nanopore” or “bottom diameter” refers to the ZMW nanopore terminus that is in contact with the substrate.

As used herein, the term “crude culture” means a biological sample that has not been subjected to extensive isolation, purification, filtration, preparation, and/or concentration procedures. In some aspects, a crude culture may comprise one or more of cells, cell debris, proteins, nucleic acids, vesicles, and other components present in a culture medium. In some aspects, a crude culture may be obtained from one or more bacterial cultures.

As used herein, the term “lipophilic fluorophore” means a hydrophobic, membrane-affinitive fluorescent dye whose molecular structure contains non-polar (e.g., alkyl) groups that drive the dye to selectively partition into lipid-rich environments, such as cell membranes, extracellular vesicle (EV) membranes, liposomes, or other phospholipid bilayers. Once embedded in the membrane, the dye emits fluorescence upon irradiation/excitation, enabling optical detection of the labeled membrane-bound structure.

### Zero-Mode Waveguide (ZMW) Arrays

Described herein are zero-mode waveguide (ZMW) arrays for monitoring populations of single extracellular vesicles (EVs) in crude cultures. The ZMW arrays described herein comprise a plurality of ZMW nanopores arranged in parallel, with each ZMW nanopore being configured to hold a single extracellular vesicle. The parallel arrangement of nanopores enables simultaneous observation of multiple single EVs, revealing heterogeneity of single EV responses that is typically masked by conventional methods.

In various aspects, each ZMW nanopore of the ZMW array has a conical frustum shape with a top diameter that is greater than a bottom diameter. This nanopore structure facilitates efficient capture and guided entry of a single EV into the confined optical detection volume. The tapered geometry promotes funneling of vesicles toward the nanopore while positioning the vesicle near the smaller bottom aperture, thereby enhancing optical confinement, reducing background, and improving signal-to-noise for single-vesicle detection.

In some embodiments, the top diameter is about 250 nm to about 350 nm and the bottom diameter is about 150 nm to about 250 nm. For example, in various embodiments, the top diameter is about 260 nm to about 340 nm, about 270 nm to about 330 nm, or about 280 nm to about 320 nm, while the bottom diameter is about 160 nm to about 240 nm, about 170 nm to about 230 nm, or about 180 nm to about 220 nm. In some embodiments, the top diameter is about 250 nm, about 275 nm, about 300 nm, about 325 nm, or about 350 nm, and the bottom diameter is about 150 nm, about 175 nm, about 200 nm, about 225 nm, or about 250 nm. In certain nonlimiting exemplary embodiments, the top diameter is about 300 nm and the bottom diameter is about 200 nm. In some embodiments, the top diameter is at least about 50 nm greater than the bottom diameter. In other embodiments, the top diameter is at least about 25 nm, at least about 75 nm, at least about 100 nm, at least about 125 nm, or at least about 150 nm greater than the bottom diameter. In some embodiments, the top diameter is about 50 nm to about 200 nm greater than the bottom diameter, about 75 nm to about 175 nm greater than the bottom diameter, or about 100 nm to about 150 nm greater than the bottom diameter.

In some embodiments, the top diameter is selected such that particles having a characteristic size greater than the top diameter are substantially excluded from entering the ZMW nanopore. For example, in some embodiments, the top diameter is selected to block or reduce entry of particles having an effective diameter greater than about 250 nm, greater than about 300 nm, or greater than about 350 nm in size. This size-based exclusion inherently excludes whole bacterial cells in crude cultures (which are typically about 1 μm or larger) from entering the optical volume, eliminating the need to isolate EVs from their parent cells prior to analysis. In certain nonlimiting exemplary embodiments, the ZMW nanopores are configured to restrict entry of particles greater than about 300 nm in size.

In some embodiments, ZMW nanopores are configured to hold ultra-small observation volumes on the order of attoliter (10⁻¹⁸ L) to zeptoliter (10⁻²¹ L) volumes. Such volumes are achieved by operating the ZMW nanopores below optical cutoff, such that excitation light decays evanescently within the nanopore and is substantially confined to a region proximate a bottom aperture of the ZMW nanopore. In some embodiments, the confined volume is determined by one or more of the bottom diameter of the ZMW nanopore, the taper angle of the conical frustum geometry of the nanopore, the metal film thickness, and the optical wavelength used for excitation.

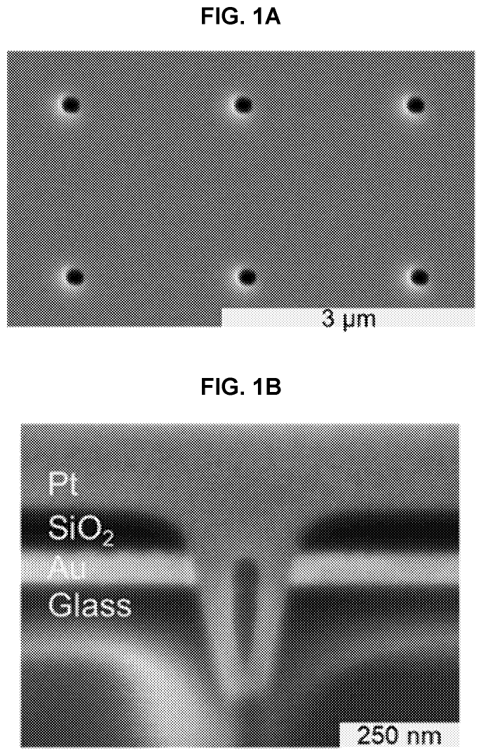

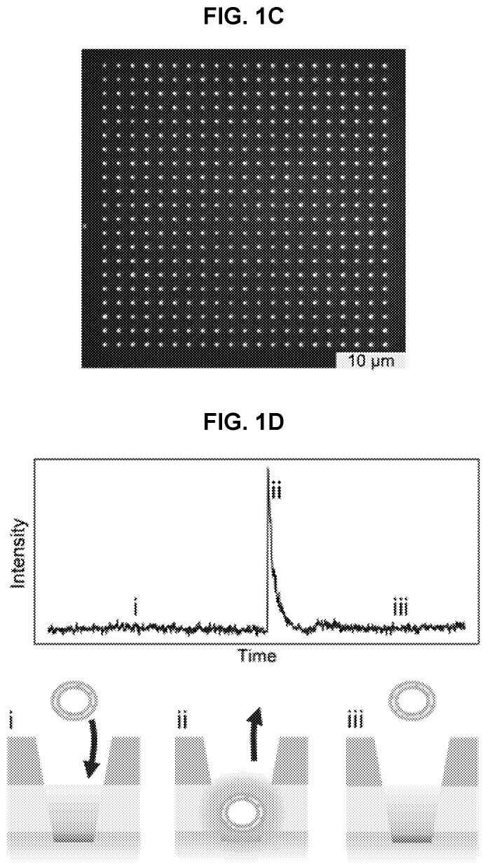

In some embodiments, the ZMW array comprises one or more layers comprising titanium (Ti), platinum (Pt), palladium (Pd), gold (Au), glass, silica (SiO₂), or combinations thereof. In some aspects, the ZMW array may be fabricated by layer-by-layer deposition followed by focused ion beam (FIB) milling. In certain nonlimiting exemplary embodiments, a titanium adhesion layer (e.g., about 5 nm to about 20 nm, such as about 10 nm) may be deposited on a glass substrate, followed by a gold layer (e.g., about 25 nm to about 100 nm, such as about 50 nm), a platinum layer (e.g., about 25 nm to about 100 nm, such as about 50 nm), and an additional titanium layer (e.g., about 2 nm to about 10 nm, such as about 5 nm). In some aspects, a silicon dioxide (SiO₂) insulating layer (e.g., about 50 nm to about 200 nm, such as about 100 nm) may be deposited on top of the metallic layers. The assembled layers may then be patterned by FIB milling to form a ZMW nanopore array. FIG. 1B and FIG. 5-6 show nonlimiting example layered configurations for ZMW nanopores as described herein.

In some aspects, the gold layer may provide biocompatibility and may contribute to embedded, annular gold nanobands within the nanopore. In some embodiments, titanium is used as an adhesion or barrier layer, while platinum, palladium, or gold provides optical attenuation, chemical stability, and compatibility with biological environments. In some embodiments, the metallic layer is supported by or deposited on a transparent substrate layer comprising glass or silica, which permits optical excitation and fluorescence collection through the substrate. In further embodiments, combinations of these materials are employed in multilayer stacks to optimize nanopore definition, fabrication yield, durability, and signal-to-noise performance during single-particle fluorescence detection.

In some embodiments, a metallic cladding layer comprising platinum, palladium, or gold has a thickness of about 30 nm to about 300 nm, about 50 nm to about 250 nm, or about 75 nm to about 200 nm, sufficient to optically block excitation light and enable operation below cutoff. For example, in certain embodiments, the metallic cladding layer has a thickness of about 100 nm, about 120 nm, about 150 nm, or about 180 nm. In some embodiments, the ZMW array further comprises one or more titanium layers serving as an adhesion layer, barrier layer, or seed layer, disposed between a metallic cladding layer and a substrate. In certain embodiments, the titanium layer has a thickness of about 1 nm to about 20 nm, about 2 nm to about 10 nm, or about 3 nm to about 7 nm, sufficient to promote adhesion without materially affecting optical performance. In some embodiments, the ZMW array is supported by a transparent substrate layer comprising glass or silica. In certain embodiments, the glass or silica substrate has a thickness of about 100 μm to about 1000 μm, about 200 μm to about 700 μm, or about 300 μm to about 500 μm. In some embodiments, the substrate has a thickness of about 500 μm.

In some embodiments, the ZMW array comprises an average interpore spacing distance of about 1.5 μm to about 2.5 μm, measured as a center-to-center distance between adjacent ZMW nanopores. For example, the average interpore spacing distance is about 1.5 μm to about 2.5 μm, about 1.6 μm to about 2.4 μm, about 1.7 μm to about 2.3 μm, about 1.8 μm to about 2.2 μm, or about 1.9 μm to about 2.1 μm. This interpore spacing may be selected to balance optical isolation, fabrication density, and parallel detection throughput, such that each ZMW nanopore functions as an independent optical observation site with minimal optical or electromagnetic cross-talk from neighboring nanopores. In some embodiments, the interpore spacing distance is sufficient to prevent overlap of evanescent optical fields, plasmonic coupling, or fluorescence bleed-through between adjacent ZMW nanopores, thereby improving signal-to-noise ratio and measurement fidelity during single-particle fluorescence detection.

In some embodiments, the ZMW array has dimensions of about 30 μm×about 30 μm to about 60 μm×about 60 μm in a regular lattice configuration (e.g., square or hexagonal packing). In some embodiments, the ZMW array has a non-square configuration with dimensions of, for example, about 30 μm×about 50 μm. In certain nonlimiting exemplary embodiments, the ZMW array has dimensions of about 40 μm×40 μm.

In some embodiments, the ZMW array comprises greater than 400 ZMW nanopores. For example, the ZMW array may comprise greater than about 425, greater than about 450, or greater than about 475 ZMW nanopores; greater than about 500, greater than about 600, or greater than about 750 ZMW nanopores; or greater than about 1000 ZMW nanopores. In other embodiments, the ZMW array may comprise less than 400 ZMW nanopores.

In various aspects, detection and monitoring of EVs using the ZMW arrays described herein may be governed by diffusion of single EV particles into the ZMW nanopore optical volume. The minimal sample preparation requirements and the ability to monitor crude culture samples in situ may enable real-time analysis of changes in the crude culture environment. In some aspects, the ZMW arrays may be used to monitor shifts in vesicle populations in response to culture perturbations, such as exposure to antibiotics or other agents.

### Methods for Monitoring Single Extracellular Vesicles

Also described herein are methods for monitoring single extracellular vesicles in a crude culture. The methods described herein comprise labeling single extracellular vesicles in the crude culture with a lipophilic fluorophore, contacting the crude culture with a ZMW array comprising a plurality of ZMW nanopores arranged in parallel, and detecting fluorescence from single extracellular vesicles positioned within the ZMW nanopores. Each ZMW nanopore is configured to hold a single extracellular vesicle, wherein single extracellular vesicles diffuse into the ZMW nanopores. In some embodiments, the methods allow for a plurality of single extracellular vesicles to be detected and monitored simultaneously in real-time.

In various aspects, the crude culture comprises one or more of cells, cell debris, proteins, nucleic acids, vesicles, and other components present in a culture medium. In some embodiments, the crude culture may comprise a bacterial crude culture, including Gram-positive bacteria, Gram-negative bacteria, or combinations thereof. In some embodiments, the crude culture comprises bacteria selected from Pseudomonas spp., Staphylococcus spp., Escherichia spp., Bacillus spp., Streptococcus spp., or combinations thereof. In some aspects, the bacterial crude culture may comprise Pseudomonas aeruginosa, Staphylococcus aureus, or combinations thereof. Such crude cultures may include whole cells, cell fragments, extracellular vesicles, secreted biomolecules, and culture media components, without prior purification, enrichment, or fractionation. In some embodiments, the crude culture may comprise mixed bacterial populations, enabling analysis of vesicle heterogeneity arising from different bacterial species within the same sample. In some embodiments, the crude culture comprises pathogenic bacteria, opportunistic pathogens, commensal bacteria, or combinations thereof. In certain embodiments, the crude culture comprises bacteria associated with clinical infections, biofilm formation, or environmental reservoirs, thereby enabling characterization of extracellular vesicles under physiologically or clinically relevant conditions. In certain preferred embodiments, bacteria in the crude culture are restricted from entering the ZMW nanopores based on size.

In various aspects, contacting the crude culture with the ZMW array comprises adding the crude culture directly to the ZMW array and applying an electrokinetic force across the ZMW array. In certain preferred embodiments, the method does not include isolating extracellular vesicles from the crude culture prior to contacting the crude culture with the ZMW array. In some embodiments, contacting may comprise depositing, dispensing, or otherwise introducing the crude culture into fluidic communication with the ZMW array (e.g., into a reservoir, chamber, or microfluidic channel positioned above the ZMW array) and then applying an electrokinetic force across the ZMW array to facilitate fluid flow and the motion of particles (including extracellular vesicles) toward and/or into individual ZMW nanopores.

In some embodiments, the electrokinetic force is produced by an electric potential applied between a first electrode disposed on a first side of the ZMW array (e.g., in a sample reservoir) and a second electrode disposed on an opposing side (e.g., beneath a substrate layer or in a second reservoir), thereby generating an electric field through the liquid volume of crude culture proximate to the ZMW nanopores. In some embodiments, the electrokinetic force may promote one or more of, for example, electrophoresis (translation of charged vesicles), electroosmosis (bulk fluid motion), and/or dielectrophoresis (motion in a non-uniform field), such that single extracellular vesicles are delivered to and transiently positioned within the attoliter/zeptoliter observation volumes of the ZMW nanopores for fluorescence interrogation. In some embodiments, detection of extracellular vesicles may be governed by diffusion of single extracellular vesicle particles into the ZMW nanopore optical volume without requiring any external pressure to drive flow. In some embodiments, the applied electrical potential difference between the first electrode and the second electrode may be about 10 mV to about 5 V, about 50 mV to about 2 V, or about 100 mV to about 1 V. In some embodiments, the electrokinetic force may be applied across the ZMW array for a duration of about 0.1 s to about 300 s, about 1 s to about 120 s, or about 5 s to about 60 s.

In various aspects, the extracellular vesicles have an average size of about 100 nm to about 400 nm. For example, in some embodiments, the extracellular vesicles have an average size of about 120 nm to about 380 nm, about 150 nm to about 350 nm, or about 180 nm to about 320 nm. In some embodiments, the extracellular vesicles have an average size of about 200 nm to about 300 nm, about 210 nm to about 290 nm, or about 225 nm to about 275 nm. In some embodiments, the extracellular vesicles have an average size of about 100 nm to about 200 nm, about 150 nm to about 250 nm, or about 250 nm to about 400 nm. In other embodiments, the extracellular vesicles may have an average size less than 100 nm or greater than 400 nm.

In various aspects, the size of extracellular vesicles is measured using one or more particle sizing techniques suitable for nanoscale objects. In some embodiments, extracellular vesicle size is determined by dynamic light scattering (DLS), wherein fluctuations in scattered light intensity arising from Brownian motion of vesicles in suspension are analyzed to calculate an average hydrodynamic diameter. DLS measurements may provide a number-weighted, intensity-weighted, or volume-weighted mean size, and are particularly useful for assessing average vesicle size and polydispersity in bulk samples. In other embodiments, vesicle size may additionally or alternatively be measured using nanoparticle tracking analysis (NTA), electron microscopy (e.g., transmission electron microscopy or scanning electron microscopy), atomic force microscopy, or fluorescence-based single-particle methods.

In various aspects, single extracellular vesicles positioned within the ZMW nanopores may have an average residence time of about 1 s to about 10 s within the confined optical observation volume, such as about 2 s to about 5 s, or about 2 s to about 3 s. This residence time corresponds to the duration over which a single vesicle remains positioned within a single ZMW nanopore in a manner sufficient to permit fluorescence excitation and detection prior to exiting the nanopore by diffusion, drift, or other transport mechanisms. The residence time may be inferred from peak width measurements during fluorescence detection. In some embodiments, an optimal residence time may be selected to balance temporal signal integration with throughput, such that individual vesicles can be reliably detected without excessive dwell times that would limit sampling of a plurality of vesicles. In some embodiments, residence time may vary based on extracellular vesicle size, with larger vesicles exhibiting longer residence times due to increased interactions with the ZMW nanopore walls. In some embodiments, smaller extracellular vesicles may present a smaller surface area for interaction with the nanopore interior, resulting in shorter residence times.

In various aspects, the lipophilic fluorophore comprises any hydrophobic, membrane-affinitive fluorescent dye that selectively partitions into lipid-rich environments, such as extracellular vesicle membranes. Once embedded in the membrane, the dye emits fluorescence upon irradiation/excitation, enabling optical detection of the labeled membrane-bound lipophilic fluorophore. In various aspects, single extracellular vesicles labeled with the lipophilic fluorophore only fluoresce upon irradiation when positioned within the ZMW nanopores. In some embodiments, the lipophilic fluorophore may comprise 3,3′-dioctadecyloxacarbocyanine (DiO), 1,1′-dioctadecyl-3,3,3′, 3′-tetramethylindocarbocyanine perchlorate (DiI), 3,3′-dioctadecyloxacarbocyanine perchlorate, 1,1′-dioctadecyl-3,3,3′,3′-tetramethylindodicarbocyanine perchlorate (DiD), 1,1′-dioctadecyl-3,3,3′,3′-tetramethylindotricarbocyanine iodide (DiR), PKH26, PKH67, Nile Red, BODIPY-based lipophilic dyes, or combinations thereof.

In various aspects, detecting fluorescence from single extracellular vesicles positioned within the ZMW nanopores comprises irradiating the ZMW array and performing fluorescence microscopy. In some embodiments, the fluorescence microscopy may comprise wide-field fluorescence microscopy, epi-illumination microscopy, or a combination thereof. The ZMW array may be illuminated uniformly, and fluorescence signals from individual nanopores may be resolved spatially using a camera-based detector. In some embodiments, optical filters, dichroic mirrors, and wavelength-selective elements may be used to discriminate fluorescence emission from excitation light and, where applicable, to distinguish between multiple lipophilic fluorophores. In some embodiments, fluorescence may be collected through a dichroic filter and imaged on a detector, such as an electron-multiplied charge coupled device (EMCCD) camera. In some embodiments, timelapse fluorescence imaging may also be used to track events of single vesicles entering and leaving across the nanopore array over time.

In various aspects, the ZMW array is irradiated with excitation light at one or more wavelengths selected to excite a lipophilic fluorophore associated with extracellular vesicles, and emitted fluorescence is collected using the fluorescence microscopy. The specific wavelength selected for excitation is based on the absorption spectrum of the lipophilic fluorophore used to label the extracellular vesicles. For example, in some embodiments, the ZMW array may be irradiated with excitation wavelengths ranging from about 400 nm to about 800 nm, including from about 400 nm to about 700 nm, about 400 nm to about 600 nm, about 400 nm to about 500 nm, about 450 nm to about 510 nm, about 470 nm to about 500 nm, about 520 nm to about 580 nm, about 530 nm to about 560 nm, about 620 nm to about 680 nm, about 630 nm to about 660 nm, about 720 nm to about 780 nm, about 740 nm to about 760 nm, about 500 nm to about 580 nm, about 425 nm to about 475 nm, about 480 nm to about 550 nm, or about 490 nm to about 530 nm.

In one nonlimiting exemplary embodiment, the lipophilic fluorophore is 3,3′-dioctadecyloxacarbocyanine (DiO) and the ZMW array is irradiated with excitation light at a wavelength of about 458 nm.

In operation, the ZMW nanopores described herein exhibit “ON” and “OFF” fluorescence states corresponding to whether an individual nanopore is occupied by a fluorescently-labeled EV or not. Upon irradiating the ZMW array, only lipophilic fluorophores within the optical volume are excited. Consequently, a nanopore may present as an OFF or “dark” state when the nanopore is empty, then as an ON or “bright” state when an EV occupies the nanopore. The fluorescence may cease once the EV leaves the optical volume, returning the nanopore to the OFF state. This ON/OFF behavior enables detection and monitoring of single EVs as they diffuse into and out of the nanopores.

In various aspects, the methods described herein may further comprise treating the crude culture with one or more agents and monitoring changes in fluorescence detection. These methods may be used to monitor shifts in vesicle populations in response to culture perturbations, such as exposure to antibiotics or other agents, in real-time. In some embodiments, the one or more agents may comprise an antibiotic, including, but not limited to, ciprofloxacin, beta-lactams (e.g., penicillin, cephalosporins), aminoglycosides (e.g., gentamicin, tobramycin), or combinations thereof. In some embodiments, exposure to an antibiotic may induce stress-related changes in bacterial cells that alter the production, release, composition, or dynamics of extracellular vesicles in the crude culture. Monitoring fluorescence detection following treatment may therefore provide an optical readout of antibiotic response, including increases or decreases in vesicle emission, changes in vesicle size distribution inferred from entry behavior, or shifts in vesicle residence times within the ZMW nanopores. In other embodiments, the one or more agents may comprise non-antibiotic compounds, such as chemical stressors, metabolic inhibitors, membrane-active agents, signaling molecules, or environmental perturbants (e.g., changes in nutrient availability, osmolarity, and/or pH). In some embodiments, combinations of agents are applied to evaluate synergistic or antagonistic effects on vesicle release and dynamics. Treatment with an agent may be performed before, during, or after contacting the crude culture with the ZMW array, and fluorescence detection may be monitored continuously or intermittently to assess temporal changes in vesicle behavior. In some embodiments, changes in fluorescence detection include variations in event frequency, fluorescence intensity, residence time, arrival rate, or population heterogeneity of extracellular vesicles across the ZMW array.

### Kits

Also described herein are kits for monitoring single extracellular vesicles in a crude culture. The kits comprise a ZMW array comprising a plurality of ZMW nanopores arranged in parallel, each ZMW nanopore being configured to hold a single extracellular vesicle; and a detection reagent. The disclosed kits may optionally further comprise one or more of buffers and receptacles; and/or one or more of packaging, a label, or instructions for use.

In various aspects, the detection reagent comprises one or more lipophilic fluorophores for labeling extracellular vesicles. In some embodiments, the lipophilic fluorophore may comprise 3,3′-dioctadecyloxacarbocyanine (DiO), 1,1′-dioctadecyl-3,3,3′,3′-tetramethylindocarbocyanine perchlorate (DiI), 3,3′-dioctadecyloxacarbocyanine perchlorate, 1,1′-dioctadecyl-3,3,3′,3′-tetramethylindodicarbocyanine perchlorate (DiD), 1,1′-dioctadecyl-3,3,3′,3′-tetramethylindotricarbocyanine iodide (DiR), PKH26, PKH67, Nile Red, BODIPY-based lipophilic dyes, or combinations thereof. In some embodiments, the detection reagent may be provided in a suitable solvent or buffer.

In some aspects, the disclosed kits may further comprise one or more detection means. The detection means may be configured to enable qualitative and/or quantitative assessment of fluorescence signals and may be integrated with or separate from other components of the kit. In some embodiments, the detection means may comprise a fluorescence detection and imaging device. The fluorescence detection and imaging device may comprise one or more of a fluorescence microscope, an electron-multiplied charge coupled device (EMCCD) camera, dichroic filters, excitation light sources, or combinations thereof. The fluorescence microscope may be configured for widefield, confocal, or epifluorescence imaging and may include optical components adapted to excite fluorescent labels and collect emitted light.

The kits described herein may be designed for use in various laboratory or clinical settings. The kit may include all necessary components and detailed instructions to facilitate safe and effective use in these settings. The instructions may be tailored to the level of expertise expected of the user, with more detailed guidance provided for settings where less specialized personnel may be involved in preparation or use. The kits described herein may comprise protective packaging to ensure the integrity of the components during storage and transport. In some embodiments, the kit may comprise cushioning materials, such as foam inserts or bubble wrap, to protect glass vials or ampoules from breakage. The kit may comprise tamper-evident seals or packaging to ensure that the kit has not been opened or tampered with prior to use. Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. As used herein, the term “instructions” may include the address of an internet site that provides the instructions.

### EMBODIMENTS

One embodiment described herein is a zero-mode waveguide (ZMW) array for monitoring populations of single extracellular vesicles in a crude culture, the ZMW array comprising a plurality of ZMW nanopores arranged in parallel, each ZMW nanopore being configured to hold a single extracellular vesicle. In one aspect, each ZMW nanopore has a conical frustum shape with a top diameter that is greater than a bottom diameter. In another aspect, the top diameter is about 250 nm to about 350 nm and the bottom diameter is about 150 nm to about 250 nm. In another aspect, the top diameter is about 300 nm and the bottom diameter is about 200 nm. In another aspect, each ZMW nanopore is configured to restrict entry of particles greater than about 300 nm in size. In another aspect, each ZMW nanopore is configured to hold attoliter (10-18 L) to zeptoliter (10-21 L) volumes. In another aspect, the ZMW array comprises one or more layers comprising titanium, platinum, palladium, gold, glass, silica, or combinations thereof. In another aspect, the ZMW array comprises greater than 400 ZMW nanopores. In another aspect, the ZMW array comprises an average interpore spacing distance of about 1.5 μm to about 2.5 μm. In another aspect, the ZMW array has dimensions of about 40 μm×40 μm.

Another embodiment described herein is a method for monitoring single extracellular vesicles in a crude culture, the method comprising labeling single extracellular vesicles in the crude culture with a lipophilic fluorophore; contacting the crude culture with a zero-mode waveguide (ZMW) array comprising a plurality of ZMW nanopores arranged in parallel, each ZMW nanopore being configured to hold a single extracellular vesicle, wherein single extracellular vesicles diffuse into the ZMW nanopores; and detecting fluorescence from single extracellular vesicles positioned within the ZMW nanopores. In one aspect, the crude culture comprises a Gram-positive bacterium, a Gram-negative bacterium, or a combination thereof. In another aspect, the crude culture comprises Pseudomonas aeruginosa or Staphylococcus aureus. In another aspect, the extracellular vesicles have an average size of about 200 nm to about 300 nm. In another aspect, the lipophilic fluorophore comprises 3,3′-dioctadecyloxacarbocyanine (DiO), 1,1′-dioctadecyl-3,3,3′,3′-tetramethylindocarbocyanine perchlorate (DiI), 3,3′-dioctadecyloxacarbocyanine perchlorate, 1,1′-dioctadecyl-3,3,3′,3′-tetramethylindodicarbocyanine perchlorate (DiD), 1,1′-dioctadecyl-3,3,3′, 3′-tetramethylindotricarbocyanine iodide (DiR), PKH26, PKH67, Nile Red, BODIPY-based lipophilic dyes, or combinations thereof. In another aspect, single extracellular vesicles positioned within the ZMW nanopores have an average residence time of about 2 s to about 5 s. In another aspect, detecting fluorescence comprises irradiating the ZMW array and performing fluorescence microscopy. In another aspect, fluorescence microscopy comprises wide-field fluorescence microscopy, epi-illumination microscopy, or a combination thereof. In another aspect, single extracellular vesicles only fluoresce upon irradiation when positioned within the ZMW nanopores. In another aspect, contacting comprises adding the crude culture to the ZMW array and applying an electrokinetic force across the ZMW array. In another aspect, the method further comprises treating the crude culture with one or more agents and monitoring changes in fluorescence detection. In another aspect, the one or more agents comprise an antibiotic. In another aspect, bacteria in the crude culture are restricted from entering the ZMW nanopores based on size. In another aspect, a plurality of single extracellular vesicles are detected and monitored simultaneously in real-time. In another aspect, the method does not include isolating extracellular vesicles from the crude culture prior to contacting the crude culture with the ZMW array.

Another embodiment described herein is a kit for monitoring single extracellular vesicles in a crude culture, the kit comprising a zero-mode waveguide (ZMW) array comprising a plurality of ZMW nanopores arranged in parallel, each ZMW nanopore being configured to hold a single extracellular vesicle; a detection reagent; optionally, buffers and receptacles; and optionally, one or more of packaging, a label, or instructions for use. In one aspect, the detection reagent comprises one or more lipophilic fluorophores for labeling extracellular vesicles. In another aspect, the kit further comprises a detection means. In another aspect, the detection means comprises a fluorescence detection and imaging device.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Materials

Ciprofloxacin (≥98.0%) and N,N-dimethylformamide (99.8%) were purchased from Sigma Aldrich. 3,3′-Dioctadecyloxacarbocyanine perchlorate (DiO) was purchased from Thermo Fisher Scientific. Glass coverslips (Nexterion Uncoated High Performance 1.5H Coverslips, Glass D263) were purchased from Applied Microarrays. All solutions were prepared using deionized (DI) water obtained from a Millipore Milli-Q system (ρ~18.2 MQ cm).

### Preparation of Bacterial Cultures

Single colonies from LB agar plates were inoculated into Fastidious Anaerobe Broth (FAB) containing 30 mM glucose for Pseudomonas aeruginosa or Tryptic Soy Broth (TSB) for Staphylococcus aureus and shaken at 37° C. overnight (~17 hours). The bacterial strains used in this study are listed in Table 1. Supernatant was obtained following centrifugation at 10,000×g for 10 min in a Heraeus Multifuge X3R centrifuge. Supernatant was filtered through 0.45 μm sterile PVDF filters. A 100 μL aliquot of sample was placed directly in contact with the ZMW for imaging, except for the real-time analysis of vesicle formation for which 90 μL was added followed by 10 μL of ciprofloxacin to achieve a final concentration of 10 μg/mL. Culture was used directly, except for experiments related to sample preparation, for which samples were diluted 1:10 in phosphate buffered saline.

TABLE 1
Bacterial Strains

| Strain | Description |
|---|---|
| Pseudomonas aeruginosa | |
| Wild-type (WT) | Wild-type PAO1C |
| ΔpqsA | PAO1C ΔpqsA markerless |
| Staphylococcus aureus | S. aureus USA 300 LAC |

### Vesicle Characterization

The bEV particle size distributions were obtained using dynamic light scattering (Nanotrac Wave II, Microtrac) utilizing a refractive index of 1.47 for the LNPs and 1.33 for the aqueous solution. The viscosity of the solution was set to 0.911 mPa·s. The solutions were scanned five times and the reported size distributions reflect an average of those five scans.

### ZMW Fabrication

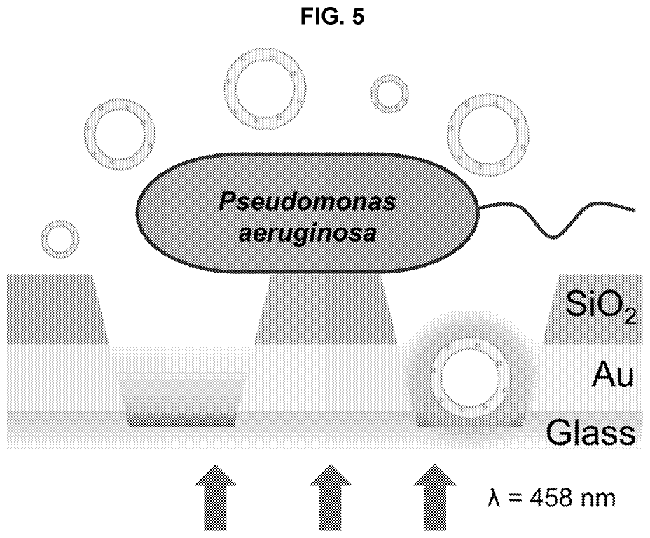

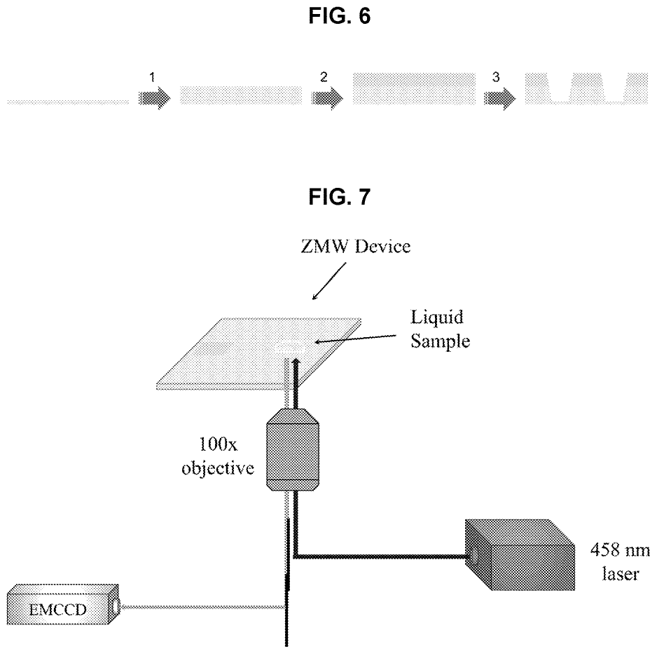

ZMWs were fabricated by layer-by-layer deposition with a shadow mask followed by focused ion beam (FIB) milling (FIG. 6). A 10 nm Ti adhesion layer was deposited on a cleanroom-quality glass slide followed by 50 nm Au, 50 nm Pt, and an additional 5 nm Ti using electron-beam evaporation (UNIVEX 450B, Oerlikon). A 100 nm SiO₂ insulating layer was then deposited using plasma-enhanced chemical vapor deposition (Unaxis 790, Plasma-Therm). The assembled layers were then patterned by Ga⁺ FIB milling (FEI, Helios G4 UX Dual Beam FIB) with 30 kV accelerating voltage, 0.75 nA ion current, and 10 ms dwell time. The resulting arrays consisted of 441 nanopores in a 40 μm×40 μm array and yielded embedded, annular Au nanobands within the nanopore.

### Fluorescence Imaging

ZMW devices were mounted on an Olympus IX-71 inverted epi-illumination microscope with a 100× objective (Olympus oil immersion, NA 1.45). Sample solution collected from the crude culture, after centrifugation, or filtration was injected directly on to the device. The nanopore array was irradiated with 458 nm excitation. Fluorescence was collected through a dichroic filter (500-575 nm) and imaged on a Peltier-cooled electron-multiplied charge coupled device (EMCCD) camera (Andor iXon) with an integration time of 0.05 s (FIG. 7).

### Image Analysis Workflow

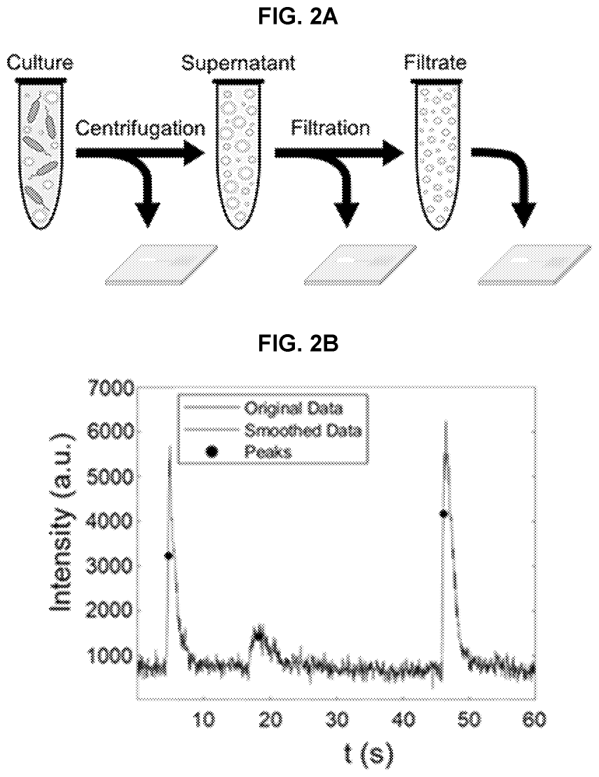

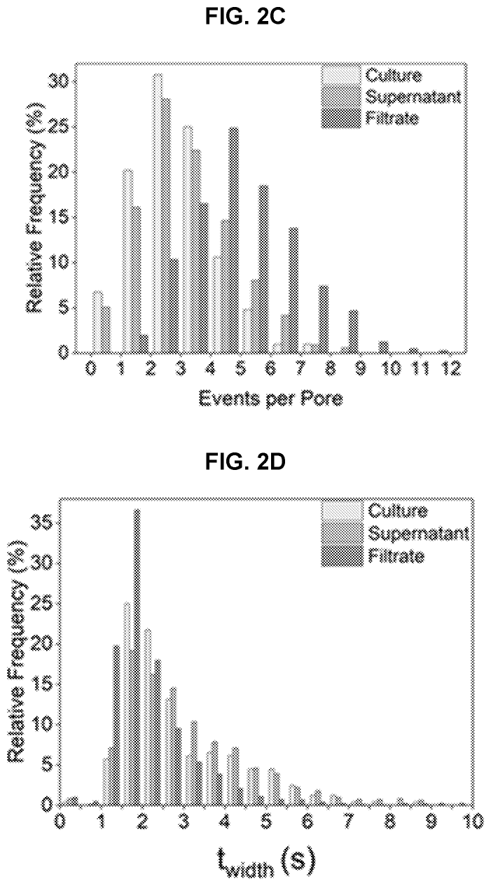

Initial image analysis was carried out as described by Sundaresan et al., Anal. Chem. 95 (35), 12993-12997 (2023). Briefly, image stacks were imported into a custom MATLAB code for pore detection, subtraction of local background, and determination of resulting intensity for each pore. Pore intensities for sets of 1200 images were analyzed using custom MATLAB code that calculated a moving average over a window of 40 time points. This average is labeled as “Smoothed Data” in FIG. 2B. A minimum peak intensity of 275 intensity units was established, and intensity features extending at or above this threshold were designated as peaks. Peak widths were measured at half peak height using custom MATLAB code.

### Design and Characterization of Zero-Mode Waveguides

ZMWs were fabricated as nano-sized conical frustums with embedded recessed-ring metallic walls, a top diameter of 300 nm, and bottom diameter of 200 nm (FIG. 1A-B) with a 2 μm interpore spacing in a 21×21 (40 μm×40 μm) array (FIG. 1C). Upon irradiating the array, only fluorophores within the optical volume are excited (FIG. 1D). Consequently, the nanopore presents as an OFF or “dark” state when the nanopore is empty (FIG. 1D, i), then as an ON or “bright” state when an EV occupies the nanopore (FIG. 1D, ii). The fluorescence ceases once the EV leaves the optical volume, returning the nanopore to the OFF state (FIG. 1D, iii).

### Bacterial EV Sample Preparation Effects

The effects of sample preparation on detection of EVs from P. aeruginosa were investigated using samples comprised of bacterial culture, culture supernatant, and culture filtrate (FIG. 2A). FIG. 5 shows an illustration of an exemplary ZMW array and method for detecting and monitoring single EVs from P. aeruginosa.

Following incubation with a lipophilic fluorophore, 3,3′-dioctadecyloxacarbocyanine iodide (DiO), samples were placed in direct contact with the ZMW for imaging. Using P. aeruginosa-derived samples, transient increases were observed in fluorescence intensity, corresponding to incorporation of single bEVs within nanopores. Intensity vs. time plots were acquired for all 441 ZMW nanopores, and the data were then smoothed using a moving 40-point average and subjected to a local threshold of 275 intensity units to identify peaks (FIG. 2B).

Even with the exclusion of large bEVs and whole bacterial cells, it is worth noting that pore-to-pore and event-to-event variations in signal intensity can be significant, as seen in FIG. 2B. Intensity is influenced by vesicle position within the pore, bEV size, and the areal density of intercalated fluorophore in the membrane. Vesicles reaching the bottom of a pore produce stronger signals than those that stop higher in the nanopore, since the evanescent field decays exponentially away from the silica-solution interface. In addition, bEVs carrying a larger fluorophore load and those in which the fluorophores are optimally placed within the evanescent field also produce larger signals. Even so, there may be additional contributions to the observed signal. For example, longer residence times can give rise to increased photobleaching. Therefore, residence time is not directly measured but inferred from peak width.

Bacterial EVs prepared by the three distinct protocols, i.e., bacterial culture, culture supernatant, and culture filtrate, were characterized with respect to event frequency, i.e., number of distinct peaks in the time trace for a given nanopore, and event duration, i.e., the width of event peaks. The event frequency distributions for bEVs prepared under all three conditions exhibited an approximately Gaussian distribution. However, a small, but statistically significant, increase in average event frequency was observed between culture and supernatant, shifting from 2.4±1.4 events per pore to 2.7±1.6 events per pore, FIG. 2C, 2E (i), averaged across the entire ensemble. A more dramatic increase was observed with filtered samples, which exhibited an ensemble average frequency of 4.6±1.8 events per pore, different than the culture and supernatant samples, statistically significant at the p≤0.001 level. In addition, the peak width, t_{width}, was also examined as an indicator of the residence time for individual bEVs in the nanopores. t_{width} Was consistent between bacterial culture and supernatant samples, FIG. 2D, 2E (ii), with ensemble average values t_{width}=2.9±0.1 s and 3.1±0.1 s, respectively. However, filtrate samples exhibited a significant decrease in t_{width} with an ensemble average of 2.20±0.02 s (p≤0.001 relative to both culture and supernatant samples).

These observed differences in bEV-nanopore occupation characteristics likely reflect underlying variations in bEV-nanopore interaction strengths among bacterial sample preparations. It is hypothesized that the sample, the cell medium, or the individual bEVs, are physically altered during centrifugation and filtration, shifting the characteristics that bEVs exhibit within the nanopores. Specifically, the viscosity of the medium and bEV size and/or structure could be affected by sample preparation methods. Both centrifugation and filtration remove viscogens which decrease the viscosity of the solution and could subsequently increase the diffusion coefficient of freely diffusing species according to the Stokes-Einstein relation, Eq. 1.

$$D = \frac{{k}_{B} T}{6 π η r}$$

where, D is the diffusion coefficient, ks is the Boltzmann constant, T is the solution temperature, η is the solution viscosity, and r is the particle radius. In this case, the removal of cells via centrifugation and further filtration of the supernatant would decrease the viscosity, thereby increasing the diffusion coefficient, consistent with the increase in event frequency observed in 2E(i). In addition to changes in viscogen populations, temperature and ionic strength, i.e., Debye length, are also likely to affect apparent bEV event frequency and residence times.

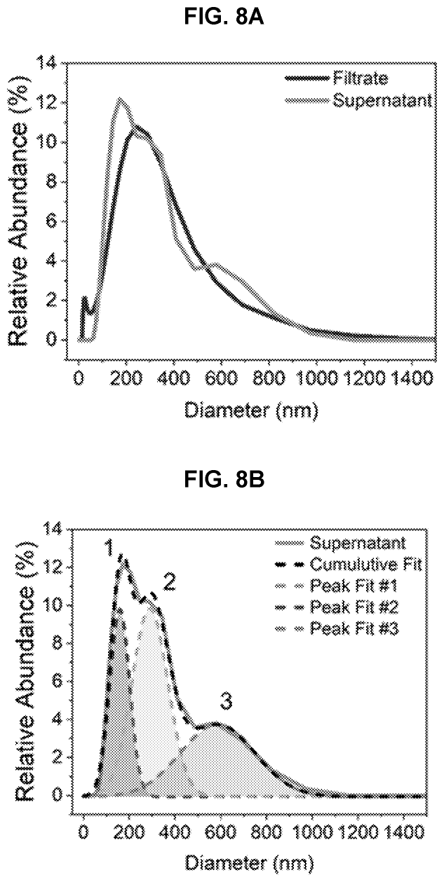

In addition to the clear trend in event frequency, residence time, as evidenced through t_{width} in FIG. 2E (ii), was observed to decrease significantly in the filtrate compared to culture and supernatant, again consistent with a larger value of D for bEVs in the filtrate. To further examine the effect of filtration on the bEVs, dynamic light scattering (DLS) measurements were made on cell supernatant and cell filtrate, FIG. 8A, and a clear difference in the DLS response of the two populations is apparent. After filtration, the population size distribution narrows, with the polydispersity index (PDI) shifting from 0.71 to 0.52, and the maximum in the bEV size distribution moving from 276±9 nm to ~247±9 nm, a statistically significant (95% confidence level) decrease in average bEV size. Furthermore, the supernatant size distribution suggests three distinct subpopulations, which collapse to a single population with a maximum near that of the second supernatant subpopulation in the filtrate (FIG. 8B). The smaller bEVs present after the sample is filtered may yield smaller t_{width} values, because they do not interact with the walls of the nanopore as strongly as larger bEVs, because the smaller bEVs present a smaller surface area with which to interact with the nanopore interior.

### Mechanism of bEV Production

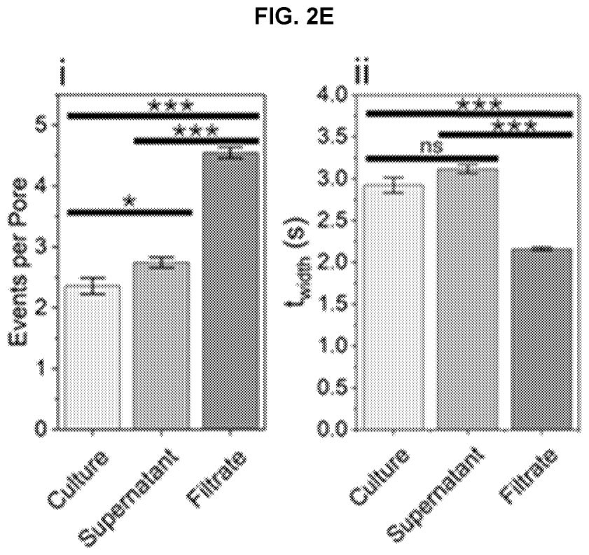

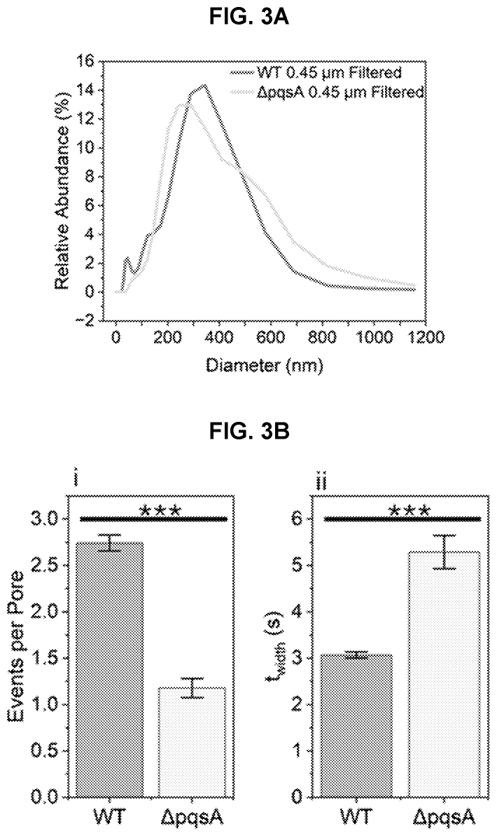

The bEV size distribution was further investigated by isolating the mechanism of bEV production. Bacterial EVs are produced by both blebbing and through cell lysis. Blebbing in P. aeruginosa produces outer membrane vesicles (~100-300 nm) while vesicles formed following explosive cell lysis are typically much larger. The small molecule, Pseudomonas Quinolone Signal (PQS), promotes blebbing and the formation of smaller vesicles in P. aeruginosa culture. Therefore, the wild type (WT) P. aeruginosa was compared to a P. aeruginosa ΔpqsA mutant strain, lacking PQS and greatly diminishing contributions from the blebbing pathway. Thus, bEVs are produced almost entirely by cell lysis in the ΔpqsA strain. Consistent with cell lysis being the dominant mechanism in the ΔpqsA strain, this strain was found to produce bEVs with a statistically significantly (95% confidence level) larger average diameter (330±13 nm) than the WT (~286±13 nm), accentuated by the presence of a large-diameter (~570 nm) subpopulation, as shown in FIG. 3A. Consistent with the DLS-derived size distributions, the larger bEVs produced by the ΔpqsA strain exhibit a much smaller number of events per pore, FIG. 3B(i), and a much larger residence time, FIG. 3B (ii).

There is a clear relationship between event frequency and bEV size in FIG. 2E (i), 3B (i), indicating that it is possible to access the effect either by sample post-treatment or by genetic manipulation. It is hypothesized that the significant decrease in event frequency observed at larger bEV sizes—either going from filtrate to supernatant or from WT to mutant—is related to more favorable bEV-nanopore interactions for larger bEVs. Thus, increasing bEV particle size is associated with longer t_{width} and less events per pore. This is consistent with previous work in which supported lipid bilayer structures form from lipid bilayer nanoparticles rupturing upon coming into contact with SiO₂ surfaces and that this phenomenon is more significant with larger vesicles diameters. This principle was used to generate supported lipid bilayers within nanopore arrays, monitoring their expansion through decreasing ionic current and fluorescence recovery after photobleaching (FRAP). It was hypothesized that a critical surface tension is reached when vesicles enter the nanopore, thus rupturing the membrane and eventually blocking the nanopore. A similar mechanism is consistent with the observations made in this work. For example, supported lipid bilayers may form on the SiO₂ portions of the nanopore walls when vesicles rupture, resulting in a lipophilic surface. This new, lipophilic surface would be expected to increase bEV-surface interactions, resulting in increased t_{width} and fewer events.

Characterization of bEVs from Gram-positive Bacteria

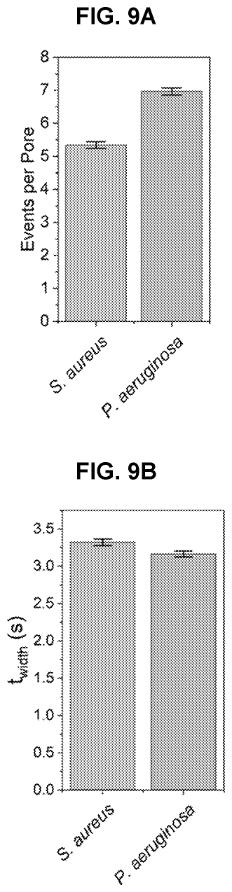

The outer membranes of Gram-negative bacteria, such as P. aeruginosa, are composed of lipopolysaccharides (LPS). LPS play a significant role in the interaction of Gram-negative bacteria with their environments, protecting them from challenges, such as osmotic pressure and antibiotics. Gram-positive bacteria, however, possess an outer, rigid peptidoglycan coat, with bEVs being produced from fractures in the outer peptidoglycan cell wall that allow blebbing of the inner cytoplasmic membrane. Staphylococcus aureus is a Gram-positive bacterium whose bEVs have promise as vaccines for sepsis. Previous studies have shown that the size range of bEVs obtained from Gram-positive and Gram-negative bacteria are comparable, but the outer surface of bEVs from Gram-positive bacteria lack LPS. To determine if the ZMW array measurement strategy developed here can recognize bEVs from both Gram-negative and Gram-positive bacteria, S. aureus filtrate was monitored on ZMWs using DiO as a fluorescent tag. As shown in FIG. 9A-B, bEVs obtained from S. aureus were captured in the nanopores on the zero-mode waveguide array similar to those obtained from P. aeruginosa, demonstrating that the parallel zero-mode waveguide array can be used to characterize bEVs from both Gram-positive and Gram-negative bacteria.

### Real-Time, In Situ Detection of Vesicle Formation

Real time detection of vesicle formation has been described using fast three-dimensional-structured illumination super-resolution microscopy. However, a high throughput method for real-time monitoring of bEV formation in P. aeruginosa culture has not yet been reported. To test the ability of ZMW nanopore arrays to monitor perturbations of bEV production in real-time and thus provide a functional assay, the effect of the addition of antibiotics to P. aeruginosa culture was examined without centrifugation or filtering. Ciprofloxacin, a fluoroquinolone antibiotic, promotes explosive cell lysis after which membrane debris undergoes subsequent bEV formation and can reduce the number of viable P. aeruginosa cells in culture by 2 orders of magnitude within 15 min. Furthermore, bEVs have been demonstrated to play a significant role in antimicrobial resistance in P. aeruginosa. For example, P. aeruginosa has been shown to utilize bEVs to combat antibiotics by transferring antibiotic resistant genes from cell-to-cell and by serving as a protective buffer on the cell membrane.

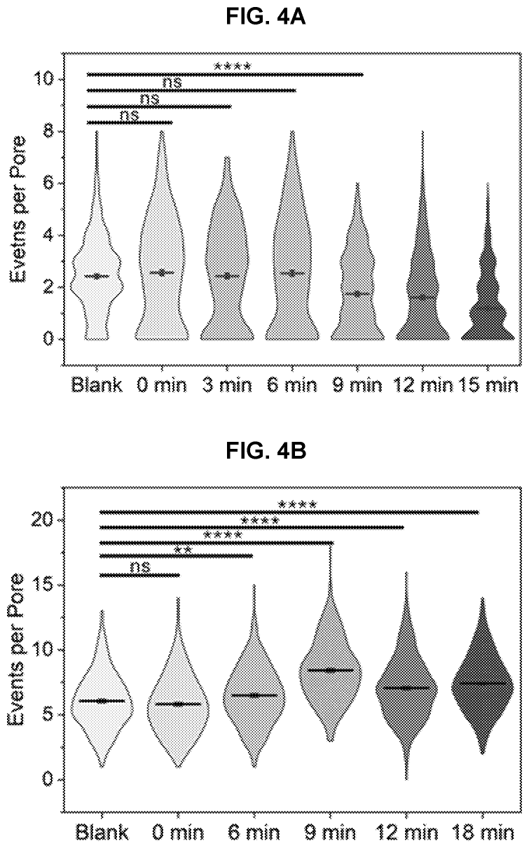

To ascertain the ability of the nanopore ZMW array to detect changes in bEV formation in real-time, undiluted P. aeruginosa culture was examined using ZMWs both without ciprofloxacin, FIG. 4A, and with the addition of ciprofloxacin, FIG. 4B. Ciprofloxacin, a fluoroquinolone antibiotic, kills bacteria by blocking DNA replication and promoting cell lysis through the RecA protein-mediated stress response. This leads to degradation of the bacterial peptidoglycan cell wall and subsequent death by explosive cell lysis, which is associated with the production of bEVs ranging from 100-800 nm in diameter with the highest proportion of vesicles 200-300 nm in diameter, therefore an increase in bEVs following treatment with this antibiotic was expected to be detected. In the absence of ciprofloxacin, FIG. 4A, the number of events per pore remains constant before decreasing from 9 to 15 min, suggesting that with the use of crude cultures, biofouling may reduce the number of detectable events at longer times. Following the addition of ciprofloxacin, a significant increase in the number of events is detected at 6 min, continuing through 9 min before eventually reaching steady state at 12-18 min, the observed rapid effect agreeing with previous work in which significant cell death occurs within 15 minutes. These data clearly establish the ability of the nanopore array assay to monitor functional changes in bEV populations caused by external events, such as exposure of the parent bacterial cells to antibiotics. Furthermore, the observed trend in number of events is consistent with the expected increase in EVs after ciprofloxacin-induced cell lysis.

In this study, single extracellular vesicles were detected from both Gram-negative and Gram-positive bacteria using ZMWs. Pores with a 300 nm diameter provide a soft threshold for vesicle capture with the probability of vesicle detection decreasing as size increases. Using a membrane specific dye, fluorescence is obtained from bEVs that enter the ZMW nanopores, while the progenitor bacterial cells are excluded. The intrinsic nanometer dimensions of the ZMW nanopores makes it possible to assemble large parallel arrays-here realized as a 21×21 array allowing simultaneous observation of 441 nanopores. Timelapse imaging was used to track single vesicles entering/leaving events in each of the 441 pores over 60 s producing 1200 images at 20 Hz acquisition frequency. Analysis of the ~5.29×10⁵ (x, y, t) regions of interest, then yielded information concerning both the frequency of vesicle uptake by the nanopores and the duration of their occupation. Vesicle populations biased toward smaller vesicles, such as those obtained from the outer membrane of P. aeruginosa, were found to produce larger average numbers of events per pore with shorter duration times, compared to bacterial samples biased toward larger vesicles (>100 nm), the latter being characterized by fewer events and longer duration times.

The ability to monitor extracellular vesicles in situ and in real time complements current methods available, which typically require elaborate preparation as well as much longer analysis times. The potential advantages of the ZMW array for characterization of EV populations were confirmed by monitoring increases in P. aeruginosa bEV production following antibiotic exposure in real time. Ciprofloxacin, which is known to increase bEV production in P. aeruginosa, was found to increase the frequency of single vesicles detected within 6 min. Future work will explore comparative studies of bEV with additional antibiotics, such as beta-lactams and aminoglycosides, which have also been shown to increase vesicle formation.

The inherent physical characteristics of small metal apertures that define ZMW behavior impose some limitations on their use for applications to EV characterization. For example, the cutoff condition, which determines the combinations of aperture size and wavelength capable of achieving zero-mode conditions, i.e., capable of frustrating far-field propagation through the ZMW nanopore, sets a size condition on EV populations that can effectively be probed. In particular, the ZMW approach is best suited to identification of relatively small vesicles rather than larger EVs, although the size cutoff is not sharp, as evidenced by the sensitivity to variations in population distributions above the 300 nm pore diameter of the nanopores used in this exemplary study.

The extent of information that can be gathered using ZMWs merits further exploration. For example, the compatibility of nanopore ZMW arrays with live cultures opens the door to many interesting experiments, where the ZMW response could be used to ascertain real time changes in EV production characteristics. The ability to detect single vesicles in a high throughput manner with minimal sample preparation positions nanopore ZMW arrays as a promising expansion to the current tool set for monitoring extracellular vesicles.

1. A zero-mode waveguide (ZMW) array for monitoring populations of single extracellular vesicles in a crude culture, the ZMW array comprising a plurality of ZMW nanopores arranged in parallel, each ZMW nanopore being configured to hold a single extracellular vesicle.

2. The ZMW array of claim 1, wherein each ZMW nanopore has a conical frustum shape with a top diameter that is greater than a bottom diameter.

3. The ZMW array of claim 2, wherein the top diameter is about 250 nm to about 350 nm and the bottom diameter is about 150 nm to about 250 nm.

4. The ZMW array of claim 2, wherein the top diameter is about 300 nm and the bottom diameter is about 200 nm.

5. The ZMW array of claim 1, wherein each ZMW nanopore is configured to restrict entry of particles greater than about 300 nm in size.

6. The ZMW array of claim 1, wherein each ZMW nanopore is configured to hold attoliter (10⁻¹⁸ L) to zeptoliter (10⁻²¹ L) volumes.

7. The ZMW array of claim 1, wherein the ZMW array comprises one or more layers comprising titanium, platinum, palladium, gold, glass, silica, or combinations thereof.

8. The ZMW array of claim 1, wherein the ZMW array comprises greater than 400 ZMW nanopores.

9. The ZMW array of claim 1, wherein the ZMW array comprises an average interpore spacing distance of about 1.5 μm to about 2.5 μm.

10. The ZMW array of claim 1, wherein the ZMW array has dimensions of about 40 μm×40 μm.

11. A method for monitoring single extracellular vesicles in a crude culture, the method comprising:
labeling single extracellular vesicles in the crude culture with a lipophilic fluorophore;
contacting the crude culture with a zero-mode waveguide (ZMW) array comprising a plurality of ZMW nanopores arranged in parallel, each ZMW nanopore being configured to hold a single extracellular vesicle, wherein single extracellular vesicles diffuse into the ZMW nanopores; and
detecting fluorescence from single extracellular vesicles positioned within the ZMW nanopores.

12. The method of claim 11, wherein the crude culture comprises a Gram-positive bacterium, a Gram-negative bacterium, or a combination thereof.

13. The method of claim 11, wherein the crude culture comprises Pseudomonas aeruginosa or Staphylococcus aureus.

14. The method of claim 11, wherein the extracellular vesicles have an average size of about 200 nm to about 300 nm.

15. The method of claim 11, wherein the lipophilic fluorophore comprises 3,3′-dioctadecyloxacarbocyanine (DiO), 1,1′-dioctadecyl-3,3,3′,3′-tetramethylindocarbocyanine perchlorate (DiI), 3,3′-dioctadecyloxacarbocyanine perchlorate, 1,1′-dioctadecyl-3,3,3′,3′-tetramethylindodicarbocyanine perchlorate (DiD), 1,1′-dioctadecyl-3,3,3′, 3′-tetramethylindotricarbocyanine iodide (DiR), PKH26, PKH67, Nile Red, BODIPY-based lipophilic dyes, or combinations thereof.

16-18. (canceled)

19. The method of claim 11, wherein single extracellular vesicles only fluoresce upon irradiation when positioned within the ZMW nanopores.

20. The method of claim 11, wherein contacting comprises adding the crude culture to the ZMW array and applying an electrokinetic force across the ZMW array.

21. The method of claim 11, further comprising treating the crude culture with one or more antibiotics and monitoring changes in fluorescence detection.

22-24. (canceled)

25. The method of claim 11, wherein the method does not include isolating extracellular vesicles from the crude culture prior to contacting the crude culture with the ZMW array.

26. A kit for monitoring single extracellular vesicles in a crude culture, the kit comprising:
a zero-mode waveguide (ZMW) array comprising a plurality of ZMW nanopores arranged in parallel, each ZMW nanopore being configured to hold a single extracellular vesicle;
a detection reagent;
optionally, buffers and receptacles; and
optionally, one or more of packaging, a label, or instructions for use.

27-29. (canceled)
